(19) United States
(12) Patent Application Publication
(10) Pub. No.: US 2014/0058108 A1
(43) Pub. Date: Feb. 27, 2014

(54) Lisuride, Terguride and Derivatives Thereof for Use in the Prophylaxis and/or Treatment of Fibrotic Changes

(71) Applicants: Reinhard Horowski, Michendorf (DE); Heinz Palla, Berlin (DE); Johannes Tack, Berlin (DE)

(72) Inventors: Reinhard Horowski, Michendorf (DE); Heinz Palla, Berlin (DE); Johannes Tack, Berlin (DE)

(73) Assignee: SINOXA PHARMA UG, Berlin (DE)

(21) Appl. No.: 13/885,058

(22) PCT Filed: Nov. 4, 2011
(86) PCT No.: PCT/EP2011/069480

§ 371 Date: Oct. 18, 2013

(30) Foreign Application Priority Data
Nov. 11, 2010 (DE) 10 2010 051 391.1
Jul. 27, 2011 (EP) 11075179.9

(51) Int. Cl. C07D 457/12

(52) U.S. Cl. CPC C07D 457/12
USPC 546/68

(57) ABSTRACT

The present invention relates to the use of 5-HT₂ receptor antagonists and in particular of 8-α-ergolines such as lisuride, terguride and the derivatives thereof as 5-HT_{2B} and 5-HT_{2A} receptor antagonists and antioxidants in preferably higher-dosed and preferably continuous use for the treatment, progression prophylaxis and general prophylaxis of organ fibroses and other pathological organ remodeling caused by mesenchymal proliferation

Subjects of this invention are lisuride, terguride and derivatives of general formula (I)

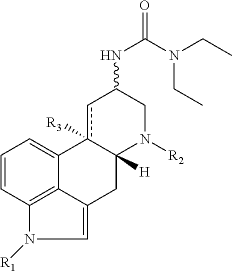

R1: Allyl, alkinyl

R2: Ethyl, n-propyl, i-propyl, allyl

R3: Hydrogen, methyl, ethyl, n-propyl, i-propyl, —CH2OH

whereby the bond between C9/C10 is either a single bond or a double bond, for use in the prophylaxis and/or treatment of fibrotic changes in organs and their vascular structure in a human or animal for impeding and/or for reversing these fibrotic changes in organs and their vascular structure. In addition, pharmaceutical formulations as well as special applications, as well as combinations with additional active ingredients and pharmaceutical formulations in combination with additional active ingredients are the subjects of this invention.

### BACKGROUND OF THE INVENTION

There are a number of diseases in which pathological fibrotic and sclerotizing changes in organs and organ systems with or without collagen deposits and pathological changes in organ structure and function result by a proliferation of connective-tissue cells and other cells of mesenchymal origin. These include primarily fibrotic organ changes with or without increased collagen formation and deposits, as are found in systemic diseases or infections (for example by HIV, Aspergillus, mycobacteria, parasites). Primary fibrosis-inducing pathological changes are found in many organs (for example, liver fibrosis, glomerulosclerosis, scleroderma, pulmonary fibrosis of many different origins, other restrictive pulmonary diseases, retroperitoneal and pleural fibroses), in part also with increased collagen production and deposits as a result of 5-HT-(=serotonin)- induced trophic effects. Also included in this respect are all forms of elevated pulmonary vascular pressure, for example as a sequela of restrictive or obstructive pulmonary diseases (for example, chronic-obstructive pulmonary disease (COPD)) as well as the right ventricular hypertrophy as a result of elevated pulmonary pressure. 5-HT can trigger these changes directly (for example released from carcinoid tumors) or secondarily (for example as a result of thrombocyte aggregation of other causes in the affected tissue with local 5-HT concentration and release).

As a sequela of the fibrosis-inducing process, the above-mentioned diseases also do not respond or respond to an only very limited extent to regional and/or systemic vasodilating medications, such as the known pulmonary vasodilators (prostacyclins, endothelin antagonists, phosphodiesterase-5 inhibitors) and systemic vasodilators. Primarily the above-mentioned pulmonary vasodilators fairly often even worsen the clinical picture [Ulrich-Somaini, S., 2009]. All of these products used to date for symptomatic treatment also have in some cases quite considerable side-effects.

Within the framework of this invention, it has now been found, surprisingly enough, that the combination of high 5-HT_{2B}- and 5-HT_{2A}-receptor affinity with strong antioxidative action has the greatest therapeutic effects. The compounds according to the invention exert their maximum effectiveness in a continuous application and from the resulting medication level that is as constant as possible.

Lisuride and terguride as well as the derivatives thereof are known for the use in the treatment of pulmonary arterial high pressure (PAH), glomerulosclerosis and secondary Raynaud's Syndrome. The effects that occur by the combination of high 5-HT_{2B}- and 5-HT_{2A}-receptor affinity presented here in connection with the antioxidative action in the same molecules were not known, however. This gives rise to quite new treatment fields and applications.

Subjects of this invention are thus lisuride, terguride and derivatives of lisuride and terguride with the general formula (I)

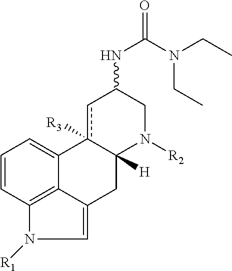

R1: Allyl, alkinyl

R2: Ethyl, n-propyl, i-propyl, allyl

R3: Hydrogen, methyl, ethyl, n-propyl, i-propyl, —CH2OH

whereby the bond between C9/C10 is either a single bond or a double bond, for use in the prophylaxis and/or treatment of fibrotic changes in organs and their vascular structure in a human or animal for impeding and/or for reversing these fibrotic changes in organs and their vascular structure.

Encompassed as subjects of this invention are in particular both 8α-enantiomers and 8β-enantiomers of the compounds according to the invention according to general formula I. In particular, 8α-lisuride and 8α-terguride as well as 8β-lisuride and 8β-terguride are thus subjects of this invention. This is surprising, since the alpha-configuration is decisive for the known dopaminergic action of the substances. A change in the configuration in the 8-position for beta-configuration means a loss of the dopaminergic action. With respect to the action on 5-HT_{2B}- and 5-HT_{2A} receptors according to the invention, however, both configurations are effective, see Table 3, Example 11. This can considerably improve the compatibility of these treatments in fibrosis-inducing diseases.

The terms “fibrotic changes in organs and their vascular structure” comprise fibrotic changes in organs and/or organ systems as well as pathological structural changes in organs and/or organ systems by mesenchymal proliferation; the term organ fibrosis is also commonly used.

Surprisingly enough, the use of lisuride, terguride and derivatives of general formula (I) according to the invention in prophylaxis and/or treatment results in extending the life of the organism. The treatment with the compounds according to the invention results in general improvement by impeding or reversing the fibrotic changes and thus in an extension of the life expectancy.

Organisms in terms of the invention are mammals, in particular human organisms, i.e., in particular humans who are suffering from fibrosis, for example.

The use in the prophylaxis and/or treatment of the above-mentioned diseases of lisuride, terguride and derivatives of general formula (I) is carried out preferably such that during the treatment time, at least 80% of the time, preferably at least 100% of the treatment time, the 5-HT_{2B}- and/or 5-HT_{2A}-receptor occupancy in the target organ is in most cases preferably complete, but at least 90%, preferably at least 95%, most preferably 100%, i.e., complete. In terms of this invention, the target organ is any fibrotic, connective tissue growth or tissue in the organism that is pathologically changed by another mesenchymal growth.

With “treatment time,” the following is meant: The receptor blocking must be carried out preferably almost completely and preferably as long as disease symptoms exist, over the entire time period, i.e., 7 days per week and 24 hours during the day. That is, 80% of the treatment time means, for example, 19.2 hours during the day.

The receptor affinity as a measurement for the blocking of the receptors of lisuride is determined in validated in-vitro systems such as the isolated pulmonary artery of the young pig (see Example 11) in a functional assay, where defined actions of 5-HT on these receptors are inhibited.

The determination of the receptor density, however, can be carried out semi-quantitatively or quantitatively, namely as follows:

Pulmonary tissue is fixed in a 4% paraformaldehyde solution and then embedded in paraffin. 3-μm sections are heated under pressure for immunohistochemistry according to manufacturer's instructions (Zymed Labs./Invitrogen, Carlsbad, Calif., USA) in 6.5 mmol of Na-citrate (pH 6.0) and incubated with antibodies against 5-HT_{2B} receptors (ab12926 of Abcam, Cambridge, UK) 1:200 and stained in sections with the Vulcan fast red Chromogen kit (Zymed) and compared to control tissue (see, for example, Dumitrascu et al., Eur. Resp. J. 37, 1104-1118, 2011).

Quantitative Reverse-Transcriptase Polymerase Chain Reaction (q RT-PCR) is carried out with RNA isolated from frozen pulmonary tissue and cDNA induced by the latter (Promega, Madison, Wi., USA) in the Mx3000P Real-Time PCR System (Stratagene, La Jolla, Calif., USA), and the receptor RNA is then quantitatively determined against a porphobilinogen reference from the same tissue (see, for example, Dumitrascu et al., see above).

The extraordinarily high 5-HT_{2B}- and 5-HT_{2A}-receptor affinity of lisuride and its derivatives and their largely uniform and quick uptake into the tissue results in a generally complete receptor blocking, as, i.a., studies with radiolabeled lisuride can show. In this case, the tissue that is to be studied before or after a lisuride treatment in the test model is prepared and homogenized, and then the specific lisuride bond is determined by a common measurement of the radioactivity in the scintillation counter. By the high receptor affinity, in this case a local concentration of these active ingredients in the pathologically changed tissue results; in particular there, the corresponding receptors are also often expressed to an increased extent. With the herewith connected locally reinforced antioxidation action, this then also results in a very specific action on the tissue pathology. The receptors themselves in this case were immunohistochemically visualized in the tissue or else quantified with, for example, RT-PCR, as described above.

High 5-HT_{2B}- and 5-HT_{2A}-receptor affinity in terms of this invention means a pA2 value of 7 (i.e., above the corresponding value for the physiological agonists 5-HT, which is on the 5-HT2b receptor at 6.5), is better at 8 and preferably at 9 or higher (see Example 11). In this case, the pA2 value reflects the negatively decade logarithm of the concentration of an antagonist, which makes it necessary to double the agonist concentration in order to restore the initial effect of the agonist without antagonists. These studies are carried out either in cloned human receptors, which are expressed on CHO cells (Newman-Tancredi et al., J. Pharmacol. Exper. Ther. 303, 815-822, 2002), or, as described in Example 11, with a functional receptor assay on pulmonary or coronary arteries in pigs (Görnemann et al., J. Pharmacol. Exp. Ther. 324, 1136-1145, 2008).

Another integral part of the invention in a preferred embodiment is that the active ingredient level of lisuride, terguide and derivatives of general formula (I) in systemic circulation of the organism during the treatment time is at least 5 pg/ml, most preferably 300-500 pg/ml, at least 80% of the time, and most preferably 100% of the treatment time continuously.

This has shown a determination of the active ingredient level by specific bioassays (for example, LC/MS/MS or radioimmunoassays) in pharmacokinetic studies on test subjects. It follows from this that a good—but in any case treatment-sufficient—correlation between the infused amount of lisuride and the thus produced plasma concentrations exists and thus also with the receptor blocking.

Also, a possible overdosage is not problematic, since lisuride and its derivatives are pure antagonists without any inherent agonistic (e.g., disease-carrying) action. In addition, there are sufficient clinical results, according to which even higher dosages, as are used for Parkinson's treatment, were well-tolerated. These considerations also hold true for other pharmaceutical forms for achieving adequately high constant plasma levels such as, for example, transdermal forms of application or high-dosed oral delayed-release formulations.

In the above-mentioned pharmacokinetic studies on test subjects, unchanged lisuride was determined selectively and with high accuracy in blood plasma by an HPLC method with mass-spectrometric detection (LC/MS/MS). The analytical determinations were performed with tert-butyl-methyl ether extracts from plasma samples, whereby in each case, 400 μl of plasma was extracted with 900 μl of tbmE, which contained the external standard proterguride (2 ng/ml). After evaporation in the organic mobile solvent acetonitrile/water (30:70)/0.1% formic acid, the extract was taken up and chromatographed with a flow rate of 300 μl/min on a C6-phenyl column by means of gradient elution (10 mmol of ammonium formate/0.1% formic acid against the above-mentioned organic mobile solvent. The detection was done with use of a mass spectrometer (TSQ) with electrospray (ESI)-interface. Sensitivity and selectivity for lisuride (n=5) was determined with 9% standard deviation and a signal/noise ratio of 40 at a concentration of 20 pg/ml. The precision of the method was determined, for example, at a concentration of 60 pg/ml with 3% standard deviation, and the lower quantitative detection limit (LLoQ) was determined with 5 pg/ml. This means that the values and information given above on the active ingredient level in the systemic circulation of the organism during the treatment time can be determined according to such a method.

Surprisingly enough, the very high affinity of the described substances for 5-HT_{2B} receptors (antagonistic effectiveness at up to 10⁻¹⁰ M concentrations) also has an advantageous effect in that the active ingredients can accumulate primarily in the fibrotized organs, in which the 5-HT2 subreceptors are often especially strongly expressed. Since, for example, a molecule of lisuride can take up to 6 free oxygen radicals, the described substances in addition have an anti-fibrotic and anti-inflammatory action even via this mechanism. This happens especially where such an antioxidative action by enhanced receptor expression is urgently desired for therapeutic purposes.

It is also advantageous that the described 5-HT_{2B} antagonists antagonize only the elevated arterial blood pressure in the lungs, but do not influence the systemic blood pressure to a significant extent. Higher blood pressure results either from a disease and stenosis of the arteries and the arterioles and capillaries downstream therefrom; this is the case in arteriosclerotic systemic high pressure (as is determined with the commonly used manometric blood pressure measurement), but also in vascularly caused idiopathic high pressure in the lungs (here, the vascular pressure is determined by an inserted heart catheter or indirectly by echocardiography).

The second possible cause for elevated arterial pressure lies in an elevated resistance in blood-supplied organs, as is caused by, for example, organ fibroses (or else in the case of kidneys by glomerulosclerosis).

It is surprising that the substances according to the invention with 5-HT_{2B} antagonism not only antagonize the fibrosis-causing effects of 5-HT but also can produce a restructuring, i.e., a renewed “remodeling” of pathological organ structures. They thus promote the additional remodeling of normal organ structure and function. This relates, for example in the case of elevated pulmonary pressure, not only to the pulmonary vessels, but rather also to the heart, in particular the right heart. In this case, it is significant that patients with pulmonary hypertension frequently die early because of the resulting hypertrophy of the right ventricle and right-heart failure resulting therefrom, so that the described therapeutic effect of the 5-HT_{2B} antagonists can extend life. Surprisingly enough, such a remodeling effect of the described 5-HT_{2B} antagonists on the hypertrophied heart is primarily not exclusively the result of an improvement of the excessive arterial pulmonary pressure and pulmonary fibrosis but rather also its function. In this case, this is an independent therapeutic effect of the 5-HT_{2B} antagonists on the myocardial hypertrophy caused by excessive 5-HT_{2B} stimulation, an effect that can be detected even a short time after the beginning of treatment (for example, by means of echocardiography). It is possible that this ontogenetically reflects a leading role of 5-HT_{2B} receptors in the normal structure of the heart in the prenatal phase.

In the example of the heart ventricle, new studies by Villeneuve et al. [2009] have shown that 5-HT, possibly released from thrombocytes, triggers the pathological remodeling of the heart, as precedes heart failure (and death), in a decisive way, but by different pathways. In this case, Villeneuve et al. distinguish the growth of the fibroblasts of the heart, where hypertrophy-promoting cytokines and interleukins are released via 5-HT_{2B}-receptor activation, by a direct activation of cardial myoblasts. This activation by 5-HT is done via 5-HT2A receptors, at higher 5-HT concentrations but also by its uptake via a specific 5-HT-uptake mechanism directly into these cells, where 5-HT then likewise induces pathological growth and organ remodeling using the monoaminoxidase A by the formation of free radicals (“reactive oxygen species, ROS”).

In the case of the substances according to the invention, it has been shown, surprisingly enough, that, for example, lisuride, but also terguride and derivatives thereof, in addition to their strong 5-HT2B antagonistic effectiveness in concentrations of similar orders of magnitude, also are strong peripheral 5-HT2A antagonists: they thus inhibit not only the secondary thrombocyte aggregation [Glusa, E. et al., 1984] independently of their triggering, but also the direct activation and proliferation of myoblasts themselves. In addition, these substances, surprisingly enough, are extremely strong radical traps. Thus, an individual molecule of lisuride can take up to 6 free oxygen radicals, terguride up to 4. Studies on 5-HT2-induced cardiac hypertrophy have demonstrated that this process runs with the generation of oxygen radicals [Bianchi, P. et al., 2005]. When it is further taken into consideration that these substances are preferably concentrated on the latter by their unprecedentedly high 5-HT2-receptor affinity (which in turn are locally strongly expressed in the case of organ hypertrophy), this combined property thus also significantly contributes to an inhibition of pathological organ growth. Moreover, these substances also have an inflammation-inhibiting effect on all of these mechanisms, so that they are effective even in the case of inflammation-triggered organ pathology (for example even in the case of pulmonary arterial high pressure triggered by COPD or infections). Such a combination of desired action mechanisms, as in the case of the described substances, could also not have been predicted by one skilled in the art in this field. Indeed, the substances according to the invention have the effects listed below against organ fibroses, organ hypertrophies, and pathological organ remodeling. In addition to the direct effects, this also includes indirect effects of 5-HT2B-receptor antagonists with resulting anti-fibrotic and anti-proliferative action, as listed below:

Direct effects of the substances according to the invention:
1. Inhibition of 5-HT2B receptor activation, such as leads to the growth of fibroblasts and their mesenchymal pathological sequelae;
2. Inhibition of 5-HT2A-receptor activation, such as is carried out in secondary thrombocyte aggregation and 5-HT release resulting therefrom;
3. Inhibition of 5-HT2A receptors on organ-specific cells, such as are increasingly expressed, for example, on myoblasts and lead to pathological organ hypertrophy;
4. Inhibition of the production and action of free oxygen radicals (ROS), such as result in an independent mechanism for organ hypertrophy, as highly effective radical traps.

Indirect effects of the substances according to the invention:
1. Inhibiting effects on remodeling processes, such as are produced from the interaction of 5-HT2B-receptor antagonists with the 5-HT transporter and with respect to the 5-HT clearance in the lungs.
2. Inhibition of the fibrotic organ remodeling by interaction of the substances according to the invention with pro-fibrotic mediators, such as, for example, PDGF and cytokines.

Surprisingly enough, specifically in the described fibrotic diseases, the 8-α-ergolines lisuride and terguride as well as the derivatives thereof are effective, namely because of their direct antagonistic effects on trophic activation of fibroblasts, fibromyoblasts, T cells and other mesenchyme cells, such as are produced primarily by an activation of 5-HT_{2B} receptors as well as by other non-vascular mechanisms.

In this case, for the desired inhibition, fibrotic organ remodeling is primarily of importance in that in the compounds according to the invention, the described 5-HT_{2B} antagonistic effect is also combined, surprisingly enough, with strong antioxidative action, which distinguishes these substances as excellent radical traps. The combination of high 5-HT2-receptor affinity with strong antioxidative action, such as exists in lisuride and its derivatives, is surprising and of great importance, if, as consequently has been determined in recent studies, for example, the pathogenetic process of 5-HT2-induced cardiac hypertrophy runs with generation of oxygen radicals [Bianchi, P. et al., 2005] and can be antagonized by radical traps [Redout, E. M. et al., 2010]. Serotonin, however, can also generate free radicals, independently of receptors, if it is released from thrombocytes at high local concentrations. In particular, the newly found combined effect, as described above, thus contributes significantly to inhibition of pathological tissue growth, since it simultaneously inhibits different pathogenetic mechanisms.

In this connection, it is important that these new and surprising actions of lisuride and its derivatives can be achieved primarily by higher-dosed applications of active ingredients that are as continuous as possible. Such an application has already been proven to be well-tolerated, individually adjustable as required, and highly effective in continuous dopaminergic stimulation in the case of advanced Parkinson's disease [Stocchi, F. et al., 2002]. In the case of the above-mentioned, surprising new applications, the actions are not based on the known dopaminergic effects of lisuride, however, but rather on its high antagonistic action on 5-HT2B receptors [Jaehnichen, S. et al., 2005] in combination with its 5-HT2A antagonism and its strong antioxidative action [Bianchi, P. et al., 2005].

These fibrosis-inducing and proliferative pathological organ diseases are characterized in that they are produced primarily or secondarily by 5-HT (serotonin) and/or oxidative stress. They are produced primarily by the activation of trophic 5-HT receptors (generally subtypes of the 5-HT2 receptor), and often the local 5-HT concentrations (for example, from thrombocytes) are increased and/or the trophic receptors are expressed to an increased extent. In this case, it is also important that even short pulses of elevated 5-HT release (such as, for example, in the Carcinoid Syndrome) and/or short phases of oxidative stress can result in permanent pathological organ remodeling with damage to the organ function. In particular by the applications that have already proven to be of value in the case of lisuride in other indications (for example by sc infusions using portable mini-pumps, by transdermal therapeutic systems, but also other depot forms), it is possible to ensure an inhibition of the serotonin-induced trophic activation of fibroblasts, T cells and other mesenchymal cells completely and over the entire day and night and thus to prevent a possible “breakthrough or escape phenomenon” with ineffectiveness resulting therefrom. The same effect can also be reached with good compatibility by higher-dosed, oral applications and delayed-release formulations, since the 5-HT_{2B}-antagonistic effectiveness can be achieved by, for example, lisuride even at a considerably lower concentration than the known and approved use of lisuride as dopamine agonist.

This invention describes the use of 5-HT-2-receptor antagonists and especially of 8-α-ergolines such as lisuride (CAS-No.: 18016-80-3,3-(9,10-didehydro-6-methylergoline-8alpha-yl)-1,1-diethylurea), terguride (trans-dihydrolisuride) and derivatives thereof as 5-HT2B and 5-HT2A-receptor antagonists and antioxidants in higher-dosed and preferably continuous applications for treatment, progression prophylaxis, and general prophylaxis of organ fibroses, and other pathological organ remodeling caused by mesenchymal proliferation. These include primarily the secondary forms of pulmonary arterial high pressure, which can occur, for example, after COPD, infections, pulmonary fibrosis, right ventricular hypertrophy as a sequela of elevated pulmonary vascular pressure as well as the fibrotic remodeling of the liver, kidneys, skin or other organ systems.

The invention further relates to salts, enantiomers, enantiomer mixtures, diastereomers, and diastereomer mixtures, hydrates, solvates, and racemates of the above-cited compounds for the production of a pharmaceutical preparation for treatment, progression prophylaxis, and general prophylaxis of organ fibroses and other organ remodeling caused by mesenchyme activation and collagen formation. These include primarily secondary forms of pulmonary high pressure, right ventricular hypertrophy as a result of elevated pulmonary vascular pressure and other organ fibroses, as well as the fibrotic remodeling of kidneys, liver, skin or other organs.

The claimed compounds lisuride and terguride are alkaline and corresponding salts can be obtained by the addition of acid, whereby organic or inorganic acids can be used. The acids, which form this type of salt of the compounds according to general formula I, include sulfuric acid, sulfonic acid, phosphoric acid, nitrous acid, nitric acid, perchloric acid, hydrobromic acid, hydrochloric acid, formic acid, acetic acid, propionic acid, succinic acid, oxalic acid, glucuronic acid (in levorotatory and dextrorotatory form), lactic acid, malic acid, tartaric acid, (hydroxymalonic acid, hydroxypropanedicarboxylic acid), fumaric acid, citric acid, ascorbic acid, maleic acid, malonic acid, hydroxymaleic acid, pyruvic acid, phenylacetic acid, (o-, m-, p-) toluic acid, benzoic acid, p-amino-benzoic acid, salicylic acid, p-amino-salicylic acid, methylsulfonic acid, ethylsulfonic acid, hydroxymethylsulfonic acid, ethylenesulfonic acid, p-toluenesulfonic acid, naphthylsulfonic acid, naphthylaminosulfonic acid, sulfanilic acid, camphorsulfonic acid, quinic acid, o-methylmandelic acid, picric acid, (2,4,6-trinitrophenol), adipic acid, amino acids, such as, for example, methionine, tryptophans, arginine, and in particular acid amino acids such as glutamine or aspartic acid.

When acid substituents are present in the compounds, basic addition salts can also be formed, in particular with alkali metals and also with amino acids. Therefore, alkali metal salts, such as sodium salt, potassium salt, or lithium salt, or magnesium salt, calcium salt, alkylamino salts or salts with amino acids can be formed with, e.g., alkaline amino acids such as lysines.

This invention relates to the use of the strong and non-antagonizable 5-HT_{2B} antagonist lisuride, its derivatives and other molecules of comparable action for the treatment or prevention of fibrotic organ changes and for the subsequent restructuring and normalization of affected organs and organ functions.

Especially by the applications already proven in the case of lisuride in other indications (for example, by sc infusions using portable mini-pumps, by transdermal therapeutic systems, but also other depot forms), it is possible to ensure an inhibition of the trophic activation of fibroblasts, T cells and other mesenchymal cells completely and over the entire day and night and thus to prevent an intermittent activation of the 5-HT2 receptor subtypes as well as a possible “escape phenomenon” with ineffectiveness of treatment following therefrom. The same effect can also be achieved by higher-dosed oral applications and delayed-release formulations with good compatibility, since the 5-HT_{2B}-antagonistic effectiveness can be achieved, for example, by lisuride already at considerably lower concentration than the known and allowed use of lisuride as a dopamine agonist.

The above-cited and claimed active ingredients and substances are suitable for treatment, progression prophylaxis, and general prophylaxis of organ fibroses of, for example, the lungs and other organ remodeling caused by mesenchyme activation and collagen formation as well as for its normalization. That the substances of formula I for this use in organ fibroses are suitable is surprising to one skilled in the art. Previous manufacturer-approved products on the basis of lisuride (for example, Dopergine®) as prolactin reducing agents and Parkinson's agents and terguride (Teluron® in Japan) even warned against the triggering of fibrotic organ changes, such as heart valves, pleural or pericardial fibrosis as well as retropleural and retroperitoneal fibrosis, as possible undesirable side-effects of these substances, such as is also in fact known for substances with ergoline structure, for example for cabergoline, pergolide, ergotamine, and methylsergide. This knowledge has previously kept one skilled in the art from the newly found uses of the above-mentioned substances.

A new finding that is relevant to achieving the desired therapeutic action is that a very short intermittent time interval of the action is adequate for the pro-fibrotic effects of 5-HT and that therefore a continuous use of 5-HT_{2B} antagonists can best prevent or can inhibit in their progression the described organ fibroses for example by means of portable mini-pumps, transdermal systems with long-term release, implants or oral delayed-release forms.

It is known that oral lisuride and terguride in their now-approved uses can also frequently trigger to some extent significant side-effects owing to their higher dosing and quick flooding. These include, for example, the orthostatic hypotension, which can result in circulatory failure and fainting and can also interfere with activities in normal life. Therefore, the manufacturers also warn against this side-effect. Such a side-effect is extremely undesirable in the case of pulmonary arterial high pressure and other organ fibroses, and it is also dangerous in the individual case. Also, for this reason, one skilled in the art would not get the idea of using such substances therapeutically in the described indications. Regarding this, the s.c. infusion with continuous action and lower daily dose is shown to be considerably superior according to the invention.

The triggering of orthostatic hypotension and collapse by the described substances is also based on their known dopaminergic action like their previous therapeutic uses. It is also triggered like other common dopaminergic side-effects not by exceeding a critical dosage or plasma concentration but rather is caused by a widely varying, oscillating plasma level, primarily in the case of oral intake with quickly reached high peaks. In the case of extended use, it results in development of tolerance, which allows the therapeutic use in the described indications. The described side-effects can, however, be avoided to a very large extent anyway by the continuous forms of the use according to the invention, since here only inadequate fluctuations of the plasma level occur over time and also a “first-pass effect” is avoided in the liver.

One use according to the invention of lisuride, terguride and derivatives thereof as 5-HT_{2B} antagonists in the described forms of organ fibroses and comparable disease in mesenchyma is also facilitated, surprisingly enough, in that because of the higher affinity of the compounds according to the invention to the 5-HT receptor, in general lower dosages than for the known applications are therapeutically effective as dopamine agonists. This means that the general compatibility of these treatments is still more advantageous and thus also is distinguished clearly from the vasodilatory substances used to date. The use is also made very easy to use by the generally very low dosage of the described 5-HT_{2B} antagonists, whose simple metabolism and in most cases also problem-free individual capacity to be metered is made very easy to use. These properties also facilitate the combination treatment, further embodied below, with other active ingredients for the same indication or for accompanying diseases, whereby also the very simple dose matching of the lisuridine infusion is another advantage.

The claimed compounds lisuride, terguride and derivatives of formula (I) are especially suitable for the treatment or prophylaxis of pathological organ remodeling, such as is caused by 5-HT and/or by local, oxidative stress.

These effects can be especially favorably used therapeutically by a constant substance action (for example by a continuous s.c. infusion) being achieved.

The preferred application is therefore a continuous application.

The use in the prophylaxis and/or treatment of the above-mentioned diseases of lisuride, terguride and derivatives of general formula (I) is carried out preferably such that during the entire treatment time, more than 90% of the time, preferably 100% of the time, the 5-HT_{2B}- and/or 5-HT_{2A}-receptor occupancy in the target organ is almost complete, but is preferably complete.

In addition, the use in the prophylaxis and/or treatment of the above-mentioned diseases of lisuride, terguride and derivatives of general formula (I) is preferably carried out such that the active ingredient level in the systemic circulation of the organism during the treatment time, at least 80% of the time, preferably at least 90%, and most preferably 100% of the time continuously, is at least 5 pg/ml, more preferably at least 100 pg/ml, more preferably at least 200 pg/ml, and most preferably 300-500 pg/ml.

The administration of lisuride, terguride and derivatives of general formula (I) in the prophylaxis and/or treatment of the above-mentioned diseases is preferably carried out at a dose of 0.01 to 5.0 mg per day, preferably 0.15 to 3.0 mg per day, and most preferably 0.25 to 1.0 mg per day.

The administration of lisuride, terguride and derivatives of general formula (I) in the prophylaxis and/or treatment of the above-mentioned diseases is carried out preferably continuously, i.e., the active ingredient level is constant as much as possible during the entire treatment time, or primarily does not go below the above-mentioned active ingredient level during the entire treatment time.

The administration according to the invention of lisuride, terguride and derivatives of general formula (I) for use in the propylaxis and/or treatment is carried out in one of the preferred embodiments on an organism, which suffers from elevated pulmonary arterial vascular pressure (PAH). In an embodiment of the invention, the PAH is the result of a disease that is selected from the group that includes COPD, infections, right ventricular hypertrophy, right-heart failure as a sequela of pulmonary hypertension, as well as other fibrotic changes in the lungs, liver, kidneys, skin or other organ systems.

Lisuride and derivatives of lisuride of general formula (I) are most preferred as therapeutic substances and as active substances in pharmaceutical formulations, and lisuride according to formula (II), both 8α-lisuride and 8β-lisuride, is vastly preferred.

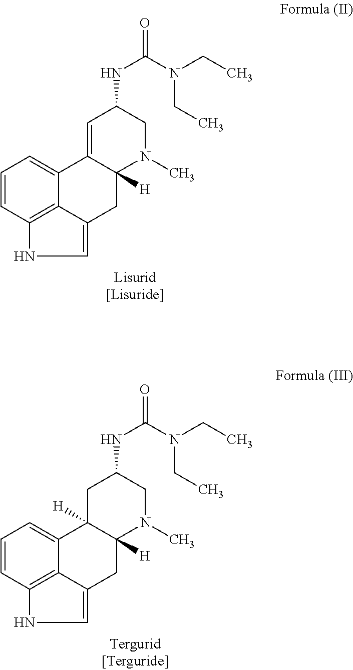

In another embodiment of the invention, terguride and derivatives of terguride of general formula (I) are preferred as therapeutic substances and as active substances in pharmaceutical formulations; terguride according to formula (III) is more preferred.

Subjects of this invention are also pharmaceutical preparations that contain lisuride, terguride and derivatives of general formula (I) for use in the prophylaxis and/or treatment of the above-described subjects of the invention.

Pharmaceutical preparations can be selected according to the invention from the group of formulations including tablets, layer tablets, coated tablets, pills, soft or hardcapsules, microcapsules, oral delayed-release dosage forms, transdermal systems, suppositories, micro- and nanocrystalline formulations, liposomal formulations, drops, nose drops, sprays, emulsions, dispersions, solutions, sterile solutions, lyophilizates, powders and inhalation aerosols.

The application or use of the pharmaceutical preparation according to the invention is preferably selected from the group that includes oral, peroral, sublingual, buccal, subcutaneous, intravenous, dermal, pulmonary or nasal use or application, whereby a subcutaneous use is most preferred.

Also, the application of pharmaceutical preparations according to the invention is preferably a continuous application.

Pharmaceutical preparations according to this invention, with an individual dose of lisuride or terguride or derivatives of general formula (I) in the range of 0.01 to 2.5 mg, are preferred, and depending on the severity of the disease, a daily dose for the patient is preferably in the range of 0.15 to 3.0 mg, most preferably in the range of 0.25 to 2.0 mg.

A sterile solution is most preferred either as a lyophilizate for preparation of a sterile solution before use or as a ready-to-use sterile solution at a dosage of 0.25 to 1.0 mg for the continuous, preferably subcutaneous, infusion at an infusion rate of 0.05 to 50 mcg/h, preferably 1 to 20 mcg/h.

In one embodiment of the invention, pharmaceutical preparations contain at least one of the claimed compounds, in particular lisuride or terguride or derivatives of general formula (I) at an individual dose of the active ingredients of 0.1 to 10 mg formulated with at least one pharmacologically compatible adjuvant, solvent, or carrier.

Pharmaceutical preparations are preferably offered as sterile solutions or lyophilizates, parenteral, peroral and oral delayed-release dosage forms, transdermal systems, microcrystalline and nanocrystalline formulations, liposomal formulations, microcapsules, emulsions, and dispersions, and they are especially suitable for subcutaneous, intravenous, dermal, transdermal, oral, peroral or pulmonary use or application.

Lactose, starch, sorbitol, mannitol, sucrose, ethyl alcohol and water can be used, for example, as pharmacologically and chemically compatible carriers, solvents or adjuvants.

In addition, starches, modified starches, gelatins, natural sugars, natural or synthetic polymers, such as, for example, acacia gum, guar, sodium alginate, carboxymethyl cellulose or polyethylene glycol, can be included as binding agents.

Cyclodextrins, modified cyclodextrins, also benzoates, chlorides, acetates, and tartrates can be included as stabilizers, and stearates, polyethylene glycol, amino acids, such as, for example, leucine, can be used as adjuvants, usually in concentrations of 0.05% to 15%.

Liquid formulations include solutions, dispersions and emulsions. Liquid preparations for parenteral use are sterile and contain water or water and solubilizers, such as, for example, propylene glycol, micelle formers and mixed micelle formers.

Starches or modified starches, alginates, aluminates, bentonites or microcrystalline cellulose can be used at concentrations of usually between 2% and 30% according to weight.

Sugar, sugar alcohols, corn, rice or potato starches, gelatins, gum arabic, tragacanth sugar, ammonium calcium alginate, carboxymethyl cellulose, hydroxypropyl methyl cellulose, polyvinyl pyrrolidone and inorganic substances can be used as adjuvants usually at concentrations of between 1% to 30% according to weight.

Pharmaceutical preparations for subcutaneous, intravenous and transdermal use just like parenteral and oral dosage forms with modified release are claimed as preferred formulations. Such formulations generally consist of a matrix, in particular a matrix with polymers, in many cases biodegradable polymers as shaping, constituting additives, in which at least one of the claimed compounds, preferably lisuride or terguride or derivatives of formula (I), is incorporated.

The polymers cited below are claimed as examples of the above-mentioned matrix-constituting polymers: polyvalerolactone, polylactides, polyglycolides, copolymers of polylactides and polyglycolides, poly-e-caprolactone, poly-hydroxybutyric acid, polyhydroxyvalerate, poly(1,4-dioxane-2,3-dione), poly(1,3-dioxan-2-one), polyanhydrides such as polymaleic acid anhydride, polyhydroxymethacrylates, fibrin, polycyanoacrylates, polycaprolactone dimethyl acrylate, poly-b-maleic acid, polycaprolactonebutyl-acrylate, multiblock polymers, such as, by way of example, oligocaprolactone diols, and oligodioxanone diols, polyether ester-multiblock polymers such as, by way of example, PEG and poly(butylene terephthalate), polypivotolactones, polycaprolactone-glycolides, poly(g-ethyl glutamates), polyorthoesters, polytrimethyl carbonates, poly-iminocarbonates, poly(N-vinyl)-pyrrolidones, polyvinyl alcohols, polyester amides, glycolated polyesters, polyphosphoesters, polyphosphazenes, poly[p-carboxyphenoxy)propane], polyhydroxypentanoic acid, polyanhydrides, polyethylene oxide-propylene oxide, polyurethanes, polyurethanes with amino acid radicals in the skeleton, polyether esters such as polyethylene oxide, polyalkylene oxalates, polyorthoesters, and their copolymers, carrageenans, fibrinogen, starches, protein-based polymers, polyamino acids, synthetic polyamino acids, zein, modified zein, polyhydroxy-alkanoates, pectinic acid, modified and non-modified fibrin and casein, carboxymethyl sulfate, albumin, in addition hyaluronic acid, heparan sulfate, heparin, chondroitin sulfate, dextran, cyclodextrins, copolymers with PEG and polypropylene glycol, gum arabic, guar, gelatins, collagen, collagen-N-hydroxysuccinimide, modifications and copolymers and/or mixtures of these substances.

Biopolymers are preferred, such as, for example, starch and denatured starch, cellulose, glycosaminoglycans, and collagen, as well as semi-synthetic and synthetic polymers such as silicones, silicone-elastomers, polydimethylsiloxane, polydimethylsiloxane containing silicon dioxide, polydimethyl siloxane containing polyalkylene oxide (Gelest®), polytetrafluoroethylene (Teflon®), polylactides, polyglycolides, polyethylene glycol, polylactide-polyglycolide copolymers, polyanhydrides, ethylene vinyl acetate polymers, poly(methyl methacrylate), cellulose ethyl ether, poly(ethyl acrylate), poly(trimethylammonium ethyl methacrylate), polydimethyl siloxanes, hydroxyethyl-polymethacrylates, polyurethanes and polystyrene-butadiene copolymers.

Moreover, peroral dosage forms with modified release as well as transdermal systems can contain microspheres or nanoparticles or microcrystals or can contain the latter as constituent components and can contain at least one of the claimed compounds, preferably terguride and lisuride. The claimed particles or crystals can, moreover, be introduced into gels and can be applied in this form and also adhere to biocompatible ceramic materials such as hydroapatite.

Within the framework of the current invention, the combination of lisuride, terguride or derivatives of formula (I) with at least one additional vasodilatory compound according to the invention is preferred.

Within the framework of the current invention, the combination of lisuride, terguride or derivatives of formula (I) with at least one additional inhibitory compound according to the invention is preferred.

Also, the application according to the invention of the above-cited combinations of lisuride, terguride or derivatives of formula (I) with the above-mentioned active ingredients according to the invention is preferably a continuous application.

Within the framework of the current invention, the combination of lisuride with at least one additional vasodilatory compound according to the invention is especially preferred. Surprisingly enough, in addition to the pressure reduction (vasodilation) in the Arteria pulmonalis, accompanying, high-level pathogenetic mechanisms, such as, for example, endothelial lesions, the release of radicals and local thrombocyte aggregations but also permissive factors, such as, for example, BMP-R2-mutations, are also thus addressed according to the invention.

Also, with the combination of lisuride with at least one vasodilatory compound, subsequent reactions of the elevated pulmonary pressure, such as hyperplasia of smooth vascular muscle cells and fibroblasts as well as fibrosis in general and ultimately a specific right-heart failure, are addressed according to the invention.

With this background, lisuride, a surprisingly advantageous combination partner, which can engage the pathogenetic cascade at various points, is preferred according to the invention. Regarding this, advantages according to the invention are:
i) As a strong peripheral 5-HT_{2A} antagonist, lisuride inhibits the thrombocyte aggregation and thus the main causes of the locally intensified 5-HT release
ii) As the strongest known 5-HT_{2B} antagonist, lisuride blocks in addition with PAH the trophic 5-HT_{2B} receptors and thus has an antifibrotic effect at the same time, thus against the progression or production of PAH
iii) As highly effective free radical traps, lisuride also antagonizes free oxygen radicals, which are increased with PAH
iv) At the same time, lisuride acts as a very strong antagonist on all alpha-adrenergic receptors and thus can the so-called Raynaud symptoms [sic], as they are frequently alleviated with PAH primarily in the case of skleroderma and primarily on the extremities; even so-called rat-bite necroses can be addressed by lisuride
v) Moreover, lisuride, as a strong 5-HT_{2A} antagonist, can also prevent or in any case weaken the pressure-induced proliferation of fibromyoblasts of the right heart, as they otherwise can lead, with PAH, to right-heart failure and ultimately to death

With the context according to the invention, the combination of lisuride, terguride and their derivatives of formula (I) as 5-HT_{2B} antagonists with known vasodilatory compounds, such as, i.a., prostacyclins and phosphodiesterase-5 antagonists, leads to surprising additive actions, preferably potentiated therapeutic actions. Also, a preferred embodiment according to the invention is the combination of lisuride, terguride and their derivatives of formula (I) as 5-HT_{2B} antagonists with inhibitory compounds of soluble guanylate cyclase. Another preferred embodiment according to the invention is the combination of lisuride, terguride and their derivatives of formula (I) as 5-HT_{2B} antagonists with inhibitory compounds of the TGF-beta-induced collagen synthesis, such as, for example, pirfenidone.

In particular, a potentiated effectiveness (not just additive) of lisuride, terguride and their derivatives of formula (I) as 5-HT_{2B} antagonists in combination with endothelin-1 antagonists, such as, i.a., bosentan, ambrisentan, larusentan, macitentan and sitaxsentan as vasodilatory compounds, is a preferred integral part of the current invention.

In this regard, the TERPAH study (personal communication of R. Reiter, A. Ghofrani) has already shown that an orally administered 5-HT_{2B} and _{2A}-antagonist terguride combination with added bosentan creates an average improvement of the pulmonary arterial pressure (PAH) by 200 dyn*sec*cm⁻⁵. In direct comparison, orally administered 5-HT_{2B} and 5-HT_{2A}-antagonist terguride combinations with a placebo only led to a pressure drop in PAH patients of between 40-70 dyn*sec*cm⁻⁵.

The especially preferred combinations of lisuride with suitable PAH medications are surprisingly effective according to the invention, since the above-mentioned effects can be added or in individual cases can be potentiated. Suitable PAH medications as combination partners, most preferably with lisuride, are selected from a group of authorized preparations such as endothelin-1 antagonists, phosphodiesterase-5 inhibitors, phosphodiesterase-4 inhibitors and prostacyclins, but also stimulators of the soluble NO-guanylate cyclase, such as riociguat and, for example, adrenomedullin (ADM). In this connection, a combination with pirfenidone, an inhibitory compound of collagen synthesis, most preferably with lisuride, is also an integral part of the current invention.

Another preferred embodiment with surprising superadditive effect follows from the combination of lisuride, terguride and their derivatives of formula (I) as 5-HT_{2B} antagonist with sildenafil and other phosphodiesterase inhibitors according to the invention as inhibitory compounds according to the invention.

In a preferred embodiment of the above-mentioned combinations, the pharmaceutically active substances according to the invention are selected from a group of lisuride, terguride and their derivatives of formula (I) as combination partners 1, administered at a dose of, for example, 0.1 to 0.6 mg of lisuride subcutaneously or 0.3 to 2.0 mg of terguride perorally per day, selected in combination with a combination partner 2 from a group of vasodilatory compounds, such as, for example, bosentan at at least 60 mg per day or, for example, sildenafil at at least 20 mg per day. In this connection, side-effects that may occur according to the invention are reduced.

Vasodilatory compounds within the context of the invention are preferably the endothelin-1 antagonists sitaxsentan, ambrisentan, larusentan, bosentan, macitentan, atrasentan, BQ-123, zibotentan, and tezosentan. Also, vasodilatory compounds within the context of the invention are phosphodiesterase-5 inhibitors such as, for example, sildenafil and phosphodiesterase-4 inhibitors, such as, for example, rolipram and prostacyclins such as, for example, iloprost, treprostinil, as well as riociguat and the peptide adrenomedullin (ADM).

Inhibitory compounds within the context of the invention are preferably pirfenidone and other inhibitors of collagen synthesis as well as imatinib and other tyrosine-kinase inhibitors.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need in order to extend life.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need, and during the treatment time, at least 80% of the time, preferably at least 100% of the treatment time, the 5-HT_{2B}- and/or 5-HT_{2A}-receptor occupancy in the target organ is at least 90%.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need, and during the entire treatment time, the 5-HT_{2B}- and/or the 5-HT_{2A}-receptor occupancy in the target organ is complete.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need, and the active ingredient level in the systemic circulation of the organism during the treatment time, at least 80% of the time, preferably 100% of the time continuously, is at least 5 pg/ml, more preferably at least 100 pg/ml, more preferably at least 200 pg/ml, and most preferably 300-500 pg/ml.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need at a dose of 0.01 to 5.0 mg per day, preferably 0.15 to 3.0 mg per day, and most preferably 0.25 to 1.0 mg per day.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered continuously to an organism in need.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered continuously to an organism in need at a daily dose of 0.01 to 5.0 mg, preferably 0.15 to 3.0 mg, and most preferably 0.25 to 2.0 mg.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need, and said organism suffers from elevated pulmonary vascular pressure (PAH).

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need, and said organism suffers from elevated pulmonary vascular pressure (PAH), which is a sequela of a disease selected from a group including CPOD, infections, right ventricular hypertrophy, and right-heart failure as a sequela of pulmonary hypertension (PAH), as well as other fibrotic changes in the lungs, liver, kidneys, skin or other organ systems.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need in combination with vasodilatory compounds.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need in combination with inhibitory compounds.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need in combination with vasodilatory compounds, i.a., selected from a group that contains sitaxsentan, ambrisentan, larusentan, bosentan, macitentan, atrasentan, BQ-123, zibotentan, tezosentan, sildenafil, iloprost, treprostinil, riociguat and adrenomedullin.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need in combination with inhibitory compounds, i.a., selected from a group that includes pirfenidone and imatinib.

Methods for the prophylaxis and/or treatment of fibrotic changes in organs and their vascular structure in a human or animal for impeding and/or for reversing said fibrotic changes in organs and their vascular structure are also subjects of this invention, whereby lisuride or terguride or a derivative of general formula (I) is administered to an organism in need in a pharmaceutical preparation.

### EXAMPLES

### Example 1

### Pharmacological Properties

### 1.A. 5-HT_{2B} Antagonism of Lisuride and Terguride

Trophic actions of serotonin mediated by 5-HT_{2B} signaling have been detected in a number of cell types, mainly in fibroblasts. They are responsible for excessive vascular remodeling processes and organ remodeling. For various compounds associated with the triggering of pathological heart valve changes and pulmonary hypertension, it is confirmed that the organ remodeling takes place as a sequela of the activation of the 5-HT_{2B} receptor, either directly or via active metabolites. These compounds include pergolide, cabergoline, fenfluramine (via the active metabolites methyl-ergonovine), MDA and MDMA (ecstasy), bromocriptine, methylsergides (via the active metabolites methyl-ergonovines) and ergotamine. Within the class of ergolines, the decisive determinant for the agonism on the 5-HT_{2B} receptor seems to lie in the β-orientation of the 8-substituents.

### 1.B. 5-HT_{2A} Antagonism of Lisuride and Terguride

Activation of HT_{2A} receptors results in thrombocyte-aggregating and vasoconstrictive effects and is connected with pro-thrombotic and hypo-fibrinolytic processes. The inhibition of the 5-HT_{2A}-mediated contraction of the coronary arteries in pigs was used to characterize the interaction of lisuride with 5-HT_{2A} receptors. In this model, lisuride also has no inherent agonistic activity at high concentrations. However, in the presence of 5-HT, lisuride inhibits the vasoconstriction with an IC50 of 1 nmol/L (see FIG. 1B).

Evaluation of the Experiment: Lisuride and its derivative terguride have a very similar pharmacological profile with identical, albeit weaker activity of terguride on the decisive 5-HT₂ receptor subtypes.

### 1C. Anti-Serotoninergic Properties of Lisuride and Terguride

An especially high anti-serotoninergic potency was detected for lisuride in vitro in isolated rat stomachs, where 5-HT activates the 5-HT_{2B} receptors [Villalon et al. 2003], and also in animal models for hyperserotonemia and 5-HT-induced effects [Podvalova, I. et al., 1972]. Also, terguride suppressed behavior abnormalities and fibrotic skin changes at the injection sites of 5-HT in rats. In the same experiment, the daily administration of 5-HT over 4 months resulted in a number of rats for development of a pulmonary valve failure, while this was not detectable in the animals treated with terguride. Analogously, terguride prevented the 5-HT-induced weight increase of the heart and liver [Hauso, O. et al., 2007].

Since lisuride and its derivative terguride have a very similar pharmacological profile with identical, albeit weaker activity of terguride on the decisive 5-HT₂ receptor subtypes, effects found in the case of terguride in this connection can also be extrapolated to lisuride.

Evaluation of the Experiment: Under physiological conditions, lisuride is a non-competitive antagonist of the 5-HT_{2A} receptor and an irreversible antagonist of the 5-HT_{2B} receptor; i.e., it cannot be antagonized itself by maximum 5-HT concentrations. The vascular reaction in the preparations of pulmonary arteries in pigs, which are pre-contracted by means of PGF2α, were used as an assay for the specific interaction with 5-HT2B receptors (see FIG. 1A). Lisuride inhibits 5-HT effects in pico- and nanomolar concentrations. This stands in good relationship to the EC50 of 5-HT for a vascular relaxation via activation of endothelial 5-HT_{2B} receptors.

### Example 2

### Antiproliferative Action of Lisuride and Terguride

Human smooth muscle cells of mesenterial origin (promo cell) were reproduced according to the manufacturer's recommendations until sealed monolayers were formed in 6 plates with PromoCell culture medium and then sowed in the same medium on 24-well trays for a cell count of 5×104 cells/batch. The cell growth was then stimulated by means of adding 10-8 mol/l of 5-HT. To determine the cell growth, 3H-thymidine (Amersham) was then added to the cell cultures, and the latter were incubated for 24 hours. After the adhesion of the cells, the culture medium was replaced by a standard medium with 0.2% fetal calf serum to stop growth and incubated again for 48 hours.

In order to test antiproliferative actions of the described substances, the cell cultures were then pre-incubated first at a concentration of 10 μmol/l of the test substances. The cell growth was then stimulated by adding 5-HT up to a final concentration of 10-8 mol/l. To measure the growth of cells, 3H-thymidine (Amersham) was then added to the cultures, and the latter was incubated for 24 hours. Then, 2 incubations were carried out in iced common salt solution with a phosphate buffer, and then a 30-minute incubation was carried out in iced 10% trichloroacetic acid at 4° C. The cells were then incubated in 0.1 molar NaOH solution (0.5 ml/incubation vessel). After neutralization with acetic acid, the 3H-thymidine uptake was measured with liquid scintillation (as 3× determination). The mean values that were found are in FIG. 2.

### Evaluation of the Experiment:

The results show that the growth, induced by 5-HT via trophic 5-HT_{2B} receptors, of mesenchymal cells of human lungs (smooth muscle cells from pulmonary vessels and bronchia or alveoles, but probably also connective-tissue fibroblasts) is quickly and effectively inhibited by the described 5-HT_{2B} antagonists. It is to be emphasized that in this model with human cells, no vasodilatory or endothelial effects can play a role (mechanisms, as they are clinically tested over time in the case of terguride in idiopathic pulmonary high pressure); rather, here, surprisingly enough, a primary action of the described substances on the proliferation of mesenchymal cells is shown.

In summary, this example describes that 5-HT_{2B} antagonists are suitable for treating conditions of pathologically increased cell proliferation and for commercial and functional restructuring of organs in non-idiopathic pulmonary high pressure and other organ fibroses.

### Example 3

### Pulmonary High Pressure

The actions of lisuride and terguride in monocrotaline-induced pulmonary high pressure in rats were studied [Reiter, R. et al., 2007]. Monocrotaline (MCT) is a toxin from plants of the Crotalaria species, which damages the endothelial cells of the pulmonary arteries after a single injection in rats, with the following hypertrophy of the smooth vascular musculature and persistent severe pulmonary high pressure. The MCT-induced pulmonary hypertension in rats is a well-established and validated model of human pulmonary hypertension, and all now-approved therapeutic agents have shown an action in this model, at least when they were used before the toxically-induced tissue remodeling.

MCT (60 mg/kg) was administered subcutaneously as a single injection in male Sprague-Dawley rats, and an identical volume of isotonic common salt solution was injected into the control animals. On days 14-28 of the experiment, either 0.25 mg/kg of lisuride or 2.5 mg/kg of terguride per day was administered via a stomach tube. In this case, the above-mentioned dose of the respective test substance was given morning and evening in a volume of 2.0 ml each to groups of respectively 6 animals, which had been treated with MCT on day 1 of the experiment. The same amount of water was fed to the control animals.

On day 28 of the experiment, two hours after the last administration of substance, the animals were narcotized with use of pentobarbital. A tracheotomy was performed, and the animals were given artificial respiration with isoflurane anesthesia. The mean arterial system pressure and the systolic pressure were measured in the right heart ventricle. After the pressure measurements, the animals were perfused with physiological common salt solution, and the right lungs were removed to determine the collagen content.

The s.c. injection of MCT, as described above, results in severe damage of the pulmonary vascular endothelium as well as excessive production of connective tissue and development of a pulmonary hypertension. Accumulation of collagen, measured as hydroxyproline content of the pulmonary tissue and increase of the right-ventricular systolic pressure reflect the described structural and functional changes.

The possible therapeutic and preventive effects of lisuride and terguride were measured in the presented model of pulmonary hypertension. In this case, under the conditions of the described experiment, the treatment with lisuride or terguride was not begun at the time of the experimentally established tissue damage but rather only 14 days after the onset of the tissue damage. According to existing literature on the experimental model, at this time, pronounced vascular changes and an increase of the right-ventricular pressure have already been demonstrated.

As Tables 1 and 2 show, in terms of a therapeutically desired effect, lisuride and terguride reduce the pathological pressure increase in the right ventricle as a measure of the elevated pulmonary pressure. As a structural correlation, a reduction of the MCT-induced increase in the hydroxyproline content of the lungs was measured, in terms of a “reverse modeling,” with treatment with lisuride and terguride.

TABLE 1
Influence of a Treatment with Lisuride or Terguride on Days 15-28 of the
Experiment on the Systolic Right-Ventricular Pressure (RVPsys) and on
the Arterial System Pressure (SAP)

| | RVPsys | SAP |
|---|---|---|
| | (mmHG) | (mmHG) |
| Control | 23 ± 4 | 118 ± 5 |
| Monocrotaline | 55 ± 5 | 114 ± 7 |
| Monocrotaline + 0.25 mg/kg of | 43 ± 7 | 109 ± 9 |
| Lisuride Bid | | |
| Monocrotaline + 2.5 mg/kg of | 39 ± 3 | 111 ± 7 |
| Terguride Bid | | |
| Results as average ± SEM (N = 6) | | |

TABLE 2
Influence of a Treatment with Lisuride or Terguride on Days 15-28 of the
Experiment on the Hydroxyproline Content of the Lungs

| | Hydroxyproline |
|---|---|
| | (μg/g of Protein) |
| Control | 1.2 ± 0.2 |
| Monocrotaline | 4.2 ± 1.1 |
| Monocrotaline + 0.25 mg/kg of Lisuride Bid | 3.3 ± 0.8 |
| Monocrotaline + 2.5 mg/kg of Terguride Bid | 2.7 ± 1.2 |
| Results as average ± SEM (N = 6) | |

Evaluation of the Experimental Results: It can be assumed that vascular effects can be detected in the monocrotaline model (for example, of prostanes), such as can be used for the treatment of pulmonary high pressure. The model does not hold true, however, as specific for this mechanism of action, as it is primarily mediated via the capillaries or arterioles of the lungs. Rather, in this model, there is also a greatly increased formation of collagen, which is produced by increasing fibroblasts and fibrocytes, i.e., cells of the mesenchyme. This is shown in the increase of the hydroxyproline level of the lungs after hydrolysis (produced to a large extent from collagen) and in the inhibition of this increase by pre-treatment with 5-HT_{2B} antagonists such as lisuride and terguide, as shown in the experiment. This speaks for an at least partial “reverse modeling,” i.e., a restoration of normal lung structures.

The same “reverse remodeling” effect can be detected in the experiment also in the right-heart chamber, and overall it thus results in a drop in the pathologically elevated pulmonary pressure. In this case, the drop/normalization of the elevated pressure in the lungs that is achieved is not, as in primary pulmonary high pressure, a first sequela of vascular effects such as, for example, after prostanes are used. Rather, the observed therapeutic effect in the case of the use of the 5-HT_{2B} antagonists is primarily the sequela of an inhibition of the mesenchymal cell proliferation primarily induced by 5-HT. This surprising finding is confirmed by the determination of an immediate inhibition of the 5-HT-induced incorporation of 3H-thymidine, an established marker of cell division and proliferation, by substances such as lisuride and terguride (described in Example 1). Here, only a lower-priority function results in a possible effect of these substances on blood capillaries or arterioles of pulmonary tissue.

This new finding now allows such substances to be used even in the case of non-vascularly produced forms of PAH and other organ diseases triggered by excessive 5-HT₂-induced proliferation and in this case to again produce normal structures and functions. This is confirmed by results of the group of Launay, which could show that an excessively increased 5-HT activity prenatally, i.e., during ontogenesis, in transgenic mice with overexpression of 5-HT_{2B} receptors leads to severe disruptions in the structure of the heart. [Nebigil, C. G., et al., 2001] It can be concluded from this that even here, a “remodeling” by 5-HT_{2B} antagonists can be of great importance for the treatment of such pathological structures. An analogous causal connection to enhanced 5-HT action optionally also holds true for observed heart deformities in newborns of women who were treated with 5-HT reuptake inhibitors because of gestational depression. Here as well, the observed heart deformities in newborns suggest that the findings are of a pathological stimulation of 5-HT_{2B} receptors.

### Example 4

### Antioxidative Action of Lisuride

If lisuride is dissolved in water, it is already measured at room temperature based on the retention times in HPL chromatograms (FIG. 3A) so that dissolved lisuride is quickly broken down into products that have a higher polarity than the initial substance itself.

Lisuride passes into solution and various reactions under the influence of light; in particular, it results in the binding of oxygen radicals. For the most part, the reaction products that can be detected by mass spectroscopy contain 2 oxygen atoms; however, compounds with 3, 4 and 5 oxygen atoms can also be detected.

A typical characteristic of mass spectra is that a peak of [M+H]++18, i.e., addition of water, or [M+H]++16, i.e., addition of an oxygen atom, has been found, as well as combinations of water and oxygen atoms. Because of the small amounts of substances contained in the samples and the short life of the reaction products, it was not possible to isolate them for structural clarification. The lisuride molecule has several positions to which water and/or an oxygen atom can be bonded (FIG. 4).

The above-described results can be of great importance, since dissolved lisuride is a strong radical trap. As already further stated above, it was demonstrated in studies on 5-HT₂-induced cardiac hypertrophy that this process proceeds with the generation of oxygen radicals [Bianchi, P. et al., 2005]. When it is further taken into consideration that these substances are preferably concentrated on the latter by their previously never reached high 5-HT₂ receptor affinity (which in turn are expressed locally enhanced in the case of organ hypertrophy), this property also contributes significantly to inhibition of pathological organ growth.

### Example 5

### Production of a Sterile Lyophilizate with Lisuride for Injection after Dissolution

1.0 g of lisuride hydrogen maleate is dissolved for injection purposes with 20 g of lactose-monohydrate, 0.4 g of citric acid monohydrate, and 1 g of sodium citrate dihydrate in 977.6 g of water. The colorless solution, which has a pH of between 4.5 and 5, is then filtered by a membrane filter and then by a sterile filter (0.2 μm) under aseptic conditions and filled to 1 g in each case in suitable vials. After sealing with a suitable plug, the solution is frozen at minus 40-50° C. and then dried in a vacuum with use of a suitable freeze-dryer, whereby in a vial, a dry cake is produced from the formulation components. Then, the vials are sealed. In this way, a batch is produced with 1,000 vials (theoretical yield) with a single dose of 1 mg of lisuride hydrogen maleate. The lyophilizate thus obtained can be reconstituted, for example, with sterile physiological common salt solution in the vial and produces a solution that is appropriate for injection for immediate use, whereby the composition of the solution with the selected adjuvants ensures adequate stability under conditions of use of at least 24 h.

### Example 6

### Production of a Sterile Lyophilizate with Terguride for Injection after Dissolution

2.0 g of terguride is dissolved for injection purposes with 20 g of lactose monohydrate, 0.4 g of citric acid monohydrate, and 1 g of sodium citrate dihydrate in 976.6 g of water. The colorless solution, which has a pH of between 4.5 and 5, is then filtered by a membrane filter and then by a sterile filter (0.2 μm) under aseptic conditions and filled to 1 g in each case in suitable vials. After sealing with a suitable plug, the solution is frozen at minus 40-50° C. and then dried in a vacuum with use of a suitable freeze-dryer, whereby in a vial, a dry cake is produced from the formulation components. Then, the vials are sealed. In this way, a batch is produced with 1,000 vials (theoretical yield) with a single dose of 2 mg of terguride. The lyophilizate thus obtained can be reconstituted with, e.g., sterile physiological common salt solution in the vial and produces a solution that is appropriate for use for injection for immediate use, whereby the composition of the solution with the selected adjuvants produces adequate stability under conditions of use of at least 24 h.

### Example 7

### Production of a Matrix Patch with Terguride for Transdermal Use

2.5 g of terguride is dissolved in 2.13 g of acetone and 51.54 g of a solution of basic butyl-methacrylate copolymer (Eudragit E 100 solution). 5 g of polyvinyl pyrrolidone (Povidone 25), 2.5 g of propylene glycol, 5 g of dodecyl(-N,N-dimethylamino acetate, alternately 1-dodecanol-n-alkyl-ether), 1 g of Foral E 105, and 0.65 g of antioxidant (butylhydroxyanisole or vitamin E) are added to the solution. The viscous solution thus obtained is continuously layered onto a polymer film that consists of, for example, polyethylene and dried under suitable process conditions with removal of the volatile solvent up to a weight per unit area of approximately 50 mg/10 cm² (±5%). This adhesive matrix is laminated with another polymer film that consists of, for example, polyethylene terephthalate, which is siliconized on one side and then punched into individual patches of 10 or 20 cm² that are suitable for the therapeutic use and packed in an air-tight and moisture-proof manner. A thus produced terguride patch releases the incorporated active ingredient continuously over several days with a release rate of between 0.1 to 0.5 μg/cm²/h to the intact human skin.

### Example 8

### Production of a Matrix Patch with Lisuride for Transdermal Use

2.5 g of lisuride is dissolved in 2.13 g of acetone and 51.54 g of a solution of basic butyl-methacrylate copolymer (Eudragit E 100 solution). 5 g of polyvinyl pyrrolidone (Povidone 25), 5 g of propylene glycol monolaurate (PGML) (as an alternative, PGML/Eutanol® (2-octyldodecanol) 10:1 or PGML/Transcutol® (diethylene glycol monoethyl ether) 10:1), 1 g of Foral E 105 and 0.65 g of antioxidant (butylhydroxyanisole or vitamin E) are added to the solution. The viscous solution thus obtained is continuously layered onto a polymer film that consists of, e.g., polyethylene and dried under suitable process conditions with removal of the volatile solvent up to a weight per unit area of approximately 50 mg/10 cm² (±5%). This adhesive matrix is laminated with another polymer film that consists of, for example, polyethylene terephthalate, which is siliconized on one side and then punched into individual patches of 5 or 10 cm² that are suitable for the therapeutic use and packed in an air-tight and moisture-proof manner. A thus produced lisuride patch releases the incorporated active ingredient continuously over several days with a release rate of between 0.1 to 0.5 μg/cm²/h to the intact human skin.

### Example 9

### Production of a Sterile Preparation with Terguride for Subcutaneous Use as an Implant

50 g of micronized terguride is mixed homogeneously with 50 g of polydimethylsiloxane, and the mixture is extracted by means of suitable standard methods, preferably by extrusion into a thread-like matrix, which is divided into pieces of 30 mm each.

An active-ingredient-free matrix is produced in the same way. In a second step, a tube-like membrane is produced with a wall thickness of, for example, 0.2 mm, also by extrusion of commercially available polydimethylsiloxane, which contains silicon dioxide, or with use of, for example, polydimethylsiloxane, which contains polyalkylene oxide (Gelest®) that is cross-linked and catalyzed with platinum. These membranes are divided into pieces 60 mm in length in each case and are allowed to swell in cyclohexane. The active ingredient-containing matrix is introduced into the tube-like membrane just like the active ingredient-free matrix, which takes place with suitable length at both ends of the tube-like membrane such that one air space each of approximately 1-3 mm remains on both sides between the active ingredient-containing and active ingredient-free matrices. Cyclohexane is removed by evaporation, and the formulation is cut to a total length of 50 mm and melted at the ends.

The product is sterilized with gas (H₂O₂ or ethylene oxide) and packed in a suitable way. In this way, an implant is produced for application under the skin, which can be localized in vivo by the air pockets by means of ultrasonic detection.

### Example 10

### Production of a Sterile Preparation with Lisuride for Subcutaneous Use as Implants

10 g of micronized terguride is mixed homogeneously with 90 g of polydimethylsiloxane, and the mixture is extracted by means of suitable standard methods, preferably by extrusion into a thread-like matrix, which is divided into pieces of 30 mm each. An active-ingredient-free matrix is produced in the same way. In a second step, a tube-like membrane is produced with a wall thickness of, for example, 0.2 mm, also by extrusion of commercially available polydimethylsiloxane, which contains silicon dioxide, or with use of, for example, polydimethylsiloxane, which contains polyalkylene oxide (Gelest®) that is cross-linked and catalyzed with platinum. These membranes are divided into pieces 60 mm in length in each case and are allowed to swell in cyclohexane. The active ingredient-containing matrix is introduced into the tube-like membrane just like the active ingredient-free matrix, which takes place with suitable length at both ends of the tube-like membrane such that one air space each of approximately 1-3 mm remains on both sides between the active ingredient-containing and active ingredient-free matrices. Cyclohexane is removed by evaporation, and the formulation is cut to a total length of 50 mm and melted at the ends.

The product is sterilized with gas (H₂O₂ or ethylene oxide) and packed in a suitable way. In this way, an implant is produced for application under the skin, which can be localized in vivo by the air pockets by means of ultrasonic detection.

### Example 11

### Affinities of Lisuride and Compounds Used to 5-HT_{2B} Receptors of Pulmonary Arteries in Pigs and with 5-HT_{2A} Receptors of Coronary Arteries in Pigs

| | 5-HT_{2B} | | 5-HT_{2A} | |
|---|---|---|---|---|
| Compound | pA₂ | n | pA₂ | n |
| Lisuride | 10.32 ± 0.10^{b} | 4 | 9.40 | 4 |
| | | | 0.05^{a,c} | |
| N″- | 8.08 ± 0.05 | 6 | 8.29 ± 0.03 | 5 |
| Monodesethyllisuride | | | | |
| 6-Norlisuride | 8.39 ± 0.12 | 4 | 8.84 ± 0.04 | 4 |
| 8β-Lisuride | 9.27 ± 0.11 | 4 | 8.54 ± 0.04 | 9 |
| Terguride | 8.87 ± 0.06 | 9 | 9.43 ± 0.08^{a} | 6 |
| N″- | 7.30 ± 0.02 | 4 | 7.82 ± 0.06 | 5 |
| Monodesethylterguride | | | | |
| 6-Norterguride | 7.11 ± 0.08 | 4 | 7.85 ± 0.04 | 5 |
| 8β-Terguride | 8.39 ± 0.09 | 4 | 8.29 ± 0.03 | 5 |
| Oxterguride | 6.42 ± 0.20 | 4 | 6.99 ± 0.04 | 4 |
| Ketanserin | — | | 8.88 ± 0.03^{c} | |
| SB204741 | 6.59 ± 0.20^{d} | 4 | — | |

^{a}= pD′2 - value (negatively decade logarithm of an antagonist concentration, which reduces the maximum effect by 50%; is used in non-competitive antagonism in contrast to pA2 (=negatively decade logarithm of the antagonist concentration, which makes it necessary to double the agonist concentration in order to restore the original agonist effect),
^{b}and ^{c}= values from Dissertation - Jähnischen, S. et al., FU Berlin, 2005,
^{d}= von Glusa, E. and Pertz, H. H., Brit. J. Pharmacol. 130, 692-698, 2000.

Ketanserin is a standard-5HT2A antagonist, SB204741 is a standard-5-HT_{2B} antagonist. Experimental conditions such as in Example 1.

### REFERENCES

1. Bianchi, P.; Pimentel, D. R.; Murphy, M. P.; Colucci, W. S.; Parini, A. (2005) A New Hypertrophic Mechanism of Serotonin in Cardiac Myocytes: Receptor-Independent ROS Generation. FASEB, J. 19, 641-643.
2. Glusa, E.; Markwardt, F. (1984) Interaction of Lisuride with Monoamine Receptors on Human Blood Platelets. Biochem. Pharmacol. 1984; 33: 493-496
3. Hauso, O.; Gustafsson, B. I.; Loennechen, J. P.; Stunes, A. K.; Nordrum, I.; Waldum, H. L. (2007) Long-Term Serotonin Effects in the Rat are Prevented by Terguride. Regulatory Peptides 143 (2007) 39-46
4. Hofmann, C.; Penner, U.; Dorow, R.; Pertz, H. H.; Jähnichen, S.; Horowski, R.; Latté, K. P.; Palla, D.; Schurad, B. (2006) Lisuride, A Dopamine Receptor Agonist with 5-HT2B Receptor Antagonist Properties: Absence of Cardiac Valvulopathy Adverse Drug Reaction Reports Supports the Concept of a Crucial Role for 5-HT2B Receptor Agonism in Cardiac Valvular Fibrosis. Clin Neuropharmacol 2006; 29: 80-86
5. Jähnichen, S.; Horowski, R.; Pertz, H. H. (2005) Agonism at 5-HT2B Receptors is Not a Class Effect of the Ergolines, Eur J Pharmacol 2005; 513: 225-228
6. Nebigil, C. G.; Hickel, P.; Messaddeq, N.; Vonesch, J.-L.; Douchet, M. P.; Monassier, L.; György, K.; Matz, R.; Andriantsitohaina, R.; Manivet, Ph.; Launay, J.-M.; Maroteaux, L. (2001) Ablation of Serotonin 5-HT2B Receptors in Mice Leads to Abnormal Cardiac Structure and Function Circulation 2001; 103: 2973-2979
7. Redout, E. M.; van der Torn, A.; Zuidwijk, M. J.; van de Kolk, C. W.; van Echtheld, C. J. A.; Musters, R. J. P.; van Hardeveld, C.; Paulus, W. J.; Simonides W. S. (2010) Antioxidant Treatment Attenuates Pulmonary Arterial Hypertension-Induced Heart Failure. Am J Physiol Heart Circ Physiol 298, H1038-H1047
8. Podvalovà, I.; Dlaba\check{c}, A. (1972) Lysenyl, A New Antiserotonin Agent Res. Clin. Stud. Headache 1972; 3: 325-334
9. Reiter, R.; Horowski, R.; Tack, J. (2007) Pharmaceutical Compositions for the Treatment of Capillary Arteriopathy. US Patent Appl Publ No. US 2009/0325997 A1
10. Stocchi, F.; Ruggieri, S.; Vacca, L.; Olanow, C. W. (2002) Prospective Randomized Trial of Lisuride Infusion Versus Oral Levodopa in Patients with Parkinson's Disease, Brain 2002; 125: 2058-2066
11. Ulrich-Somaini, S. (2009) Management der pulmonalen Hypertonie—was ist neu seit Dana Point? [Management of Pulmonary Hypertension—What is New Since Dana Point?] Kardiovaskuläre Medizin [Cardiovascular Medicine] 2009; 12(9): 245-250
12. Villalon, C. M.; Centurion, D.; Valdivia, L. F.; de Vries, P.; Saxena, P. R. (2003) Migraine: Pathophysiology, Pharmacology, Treatment and Future Trends, Curr vasc Pharmacol 2003; 1: 71-84
13. Villeneuve, C.; Caudrillier, A.; Ordener, C.; Pizzinat, N.; Parini, A.; Mialet-Perez J. (2009) Dose-Dependent Activation of Distinct Hypertrophic Pathways by Serotonin in Cardiac Cells. Am. J. Physiol Heart Circ. Physiol 297, H821-H828.

### DESCRIPTION OF FIGURES

FIG. 1: Antagonistic effects of lisuride and terguride on (A) 5-HT-induced and 5-HT_{2B}-mediated relaxing of PGF2α-pre-contracted pulmonary arteries in pigs and on the (B) 5-HT-induced contraction of 5-HT_{2A}-mediated coronary arteries in pigs [Jaehnichen S. et al. 2005].

FIG. 2: Reduction of cell proliferation under terguride and lisuride.

FIG. 3A: HPL Chromatogram a. Immediately after lisuride hydrogen maleate is dissolved in water (0.4 mg/ml) and b. After 4 hours at room temperature and daylight.

FIG. 3B: Mass spectrum of an aqueous solution of lisuride 5 hours after exposure to light; examples of [M+H]++16 and [M+H]++18 steps for water as well as one or more additional oxygen atoms are provided.

FIG. 4: Binding sites in the lisuride molecule, on which water or oxygen atoms can be stored and structural examples.

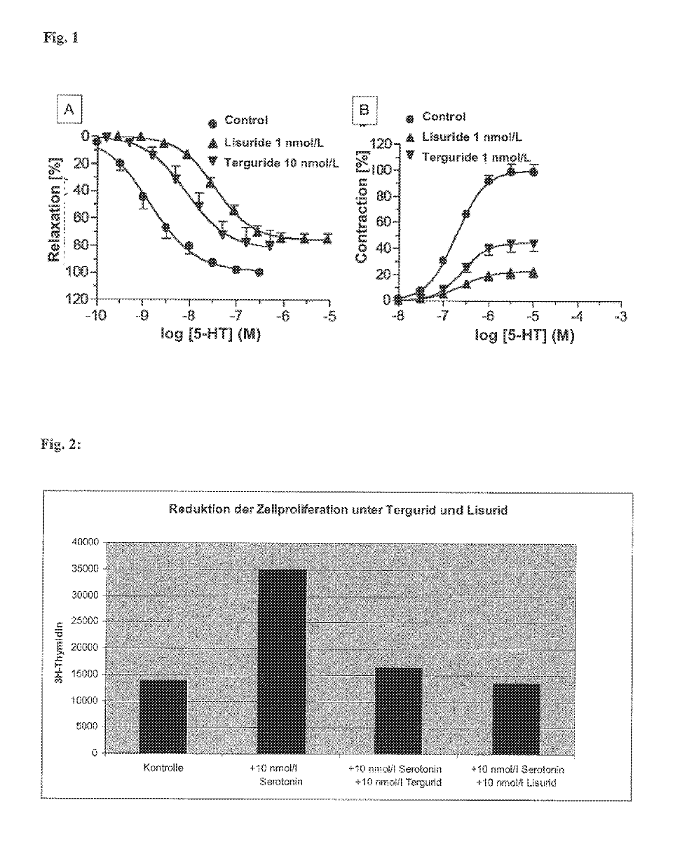

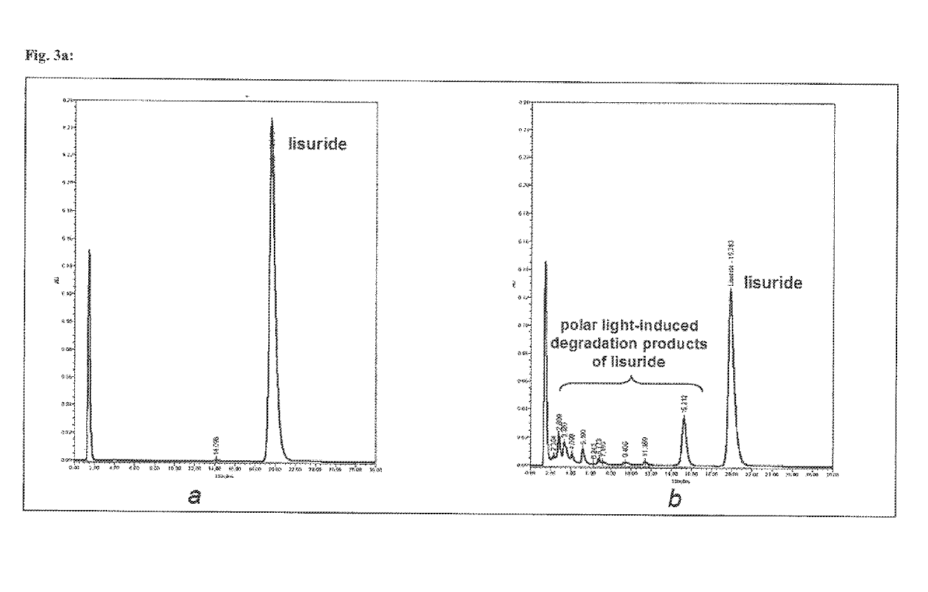

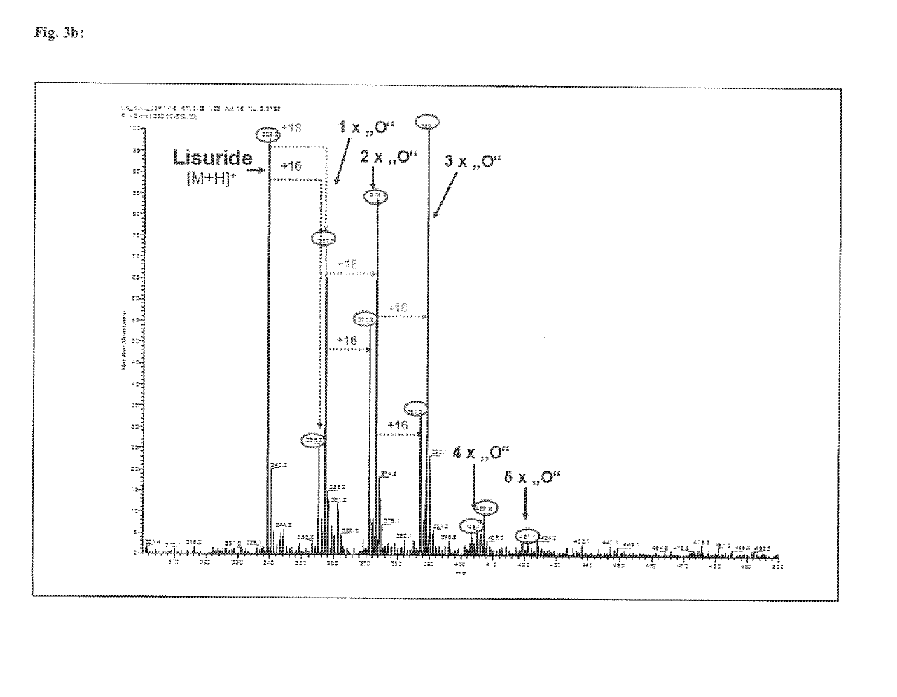

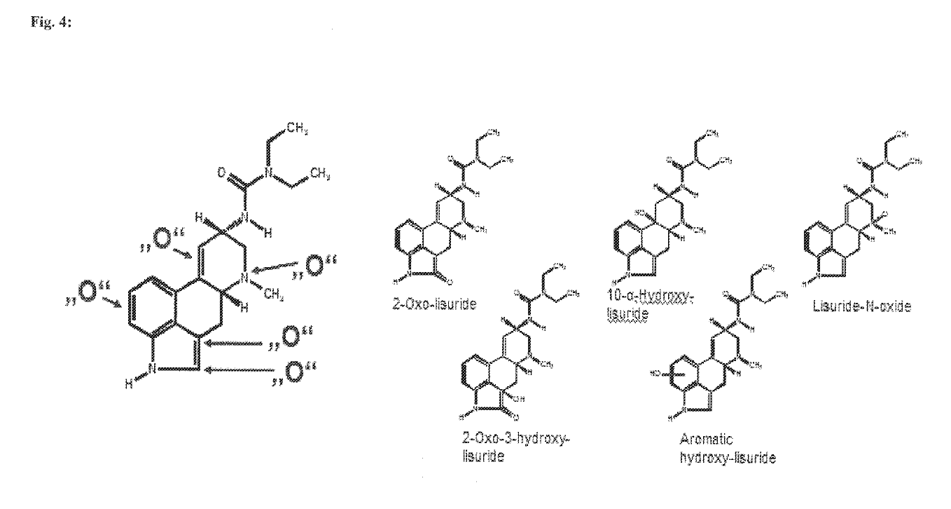

### [Key to FIG. 2:]

3H-Thymidin=3H-Thymidine
Reduktion der Zellproliferation unter Tergurid and Lisurid=Reduction of Cell
Proliferation under Terguride and Lisuride
Kontrolle=Control
Tergurid=Terguride
Lisurid=Lisuride

1. Lisuride or terguride or a derivative of general formula (I): 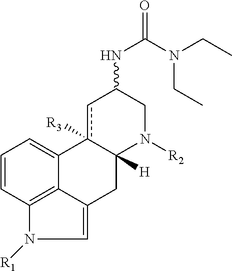
R1: Allyl, alkinyl
R2: Ethyl, n-propyl, i-propyl, alkyl
R3: Hydrogen, methyl, ethyl, n-propyl, i-propyl, —CH2OH
whereby the bond between C9/C10 is either a single bond or a double bond, for use in the prophylaxis and/or treatment of fibrotic changes in organs and their vascular structure in a human or animal for impeding and/or for reversing said fibrotic changes in organs and their vascular structure.

2. Lisuride or terguride or a derivative of general formula (I) for use in the prophylaxis and/or treatment according to claim 1 for extending the life of the organism.

3. Lisuride or terguride or a derivative of general formula (I) for use in the prophylaxis and/or treatment according to claim 1, whereby during the treatment time, at least 80% of the time, preferably at least 100% of the treatment time, the 5-HT_{2B}- and/or 5-HT_{2A}-receptor occupancy in the target organ is at least 90%.

4. Lisuride or terguride or a derivative of general formula (I) for use in the prophylaxis and/or treatment according to claim 3, whereby during the entire treatment time, the 5-HT_{2B}- and/or 5-HT_{2A}-receptor occupancy in the target organ is complete.

5. Lisuride or terguride or a derivative of general formula (I) for use in the prophylaxis and/or treatment according to claim 1, whereby the active ingredient level in the systemic circulation of the organism during the treatment time, at least 80% of the time, preferably 100% of the time continuously, is at least 5 pg/ml, more preferably at least 100 pg/ml, more preferably at least 200 pg/ml, and most preferably 300-500 pg/ml.

6. Lisuride or terguride or a derivative of general formula (I) for use in the prophylaxis and/or treatment according to claim 1, wherein the administration is carried out at a dose of 0.01 to 5.0 mg per day, preferably 0.15 to 3.0 mg per day, and most preferably 0.25 to 1.0 mg per day.

7. Lisuride or terguride or a derivative of general formula (I) for use in the prophylaxis and/or treatment according to claim 1, whereby administration is done continuously.

8. Lisuride or terguride or a derivative of general formula (I) for use in the prophylaxis and/or treatment according to claim 1, whereby administration is done continuously at a daily dose of 0.01 to 5.0 mg, preferably 0.15 to 3.0 mg, and most preferably 0.25 to 2.0 mg.

9. Lisuride or terguride or a derivative of general formula (I) for use in the prophylaxis and/or treatment according to claim 1, whereby the organism suffers from elevated pulmonary vascular pressure (PAH).

10. Lisuride or terguride or a derivative of general formula (I) for use in the prophylaxis and/or treatment according to claim 9, whereby the PAH is a sequela of a disease selected from a group including COPD, infections, right ventricular hypertrophy, and right-heart failure as a sequela of pulmonary hypertension (PAH), as well as other fibrotic changes in the lungs, liver, kidneys, skin or other organ systems.

11. Lisuride or terguride or a derivative of general formula (I) for use in the prophylaxis and/or treatment of fibrotic changes in organs and their vascular structure in a human or animal for impeding and/or for reversing said fibrotic changes in organs and their vascular structure according to claim 1, whereby said lisuride or terguride or a derivative of general formula (I) is used in combination with vasodilatory compounds.

12. Lisuride or terguride or a derivative of general formula (I) for use in the prophylaxis and/or treatment of fibrotic changes in organs and their vascular structure in a human or animal for impeding and/or for reversing said fibrotic changes in organs and their vascular structure according to claim 1, whereby said lisuride or terguride or a derivative of general formula (I) is used in combination with inhibitory compounds.

13. Lisuride or terguride or a derivative of general formula (I) for use in the prophylaxis and/or treatment according to claim 11, whereby said vasodilatory compounds, i.a., are selected from a group that includes sitaxsentan, ambrisentan, larusentan, bosentan, macitentan, atrasentan, BQ-123, zibotentan, tezosentan, sildenafil, iloprost, treprostinil, riociguat and adrenomedullin.

14. Lisuride or terguride or a derivative of general formula (I) for use in the prophylaxis and/or treatment according to claim 12, whereby said inhibitory compounds, i.a., are selected from a group that includes pirfenidone and imatinib.

15. Pharmaceutical preparation that contains lisuride or terguride or a derivative of general formula (I) according to claim 1.
